(19) United States
(12) Patent Application Publication
(10) Pub. No.: US 2006/0040002 A1
(43) Pub. Date: Feb. 23, 2006

(54) Anti-itch composition

(76) Inventor: Suzanne M. Boitano, Spring Hill, FL (US)

Correspondence Address: EDWARD M. LIVINGSTON, PA, 963 TRAIL TERRACE DRIVE, NAPLES, FL, 34103, US

(21) Appl. No.: 10/919,930

(22) Filed: Aug. 17, 2004

(51) Int. Cl. A61K 36/534; A61K 33/02

(52) U.S. Cl. 424/745; 424/747; 424/721

(57) ABSTRACT

An anti-itch composition comprising ammonium hydroxide, water, gelatin, a surfactant and menthol essential oils. The composition is a topical antiseptic preparation for neutralizing allergic reactions caused by insect bites, including ants, bees stings, fleas and the like. The composition also provides relief of itching, swelling pain, inflammation and decreases the risk of developing anaphylaxis caused by insect bites.

### BACKGROUND OF THE INVENTION

This invention relates to anti-itch compositions, more specifically, an anti-itch composition that neutralizes allergic reactions caused by red ant bites, bee stings and bug bites wherein the active ingredient is an ammonium solution.

Currently, there are many products on the market, both in the United States and worldwide, for the treatment of insect bites. Few of these products contain ammonia as the active ingredient to provide relief of itching, inflammation and antisepsis. While other products that may contain Benadryl®, hydrocortisone, benzocaine, iodide or camphor ingredients, they do not relate chemically nor reverse the allergic symptoms through an acid-base reaction principal.

Thus, the need exists for an anti-itch composition that provides not only temporary relief from the pain and itching caused by insect bites, but also provides a neutralizing effect to essentially reverse the allergic reaction.

The relevant prior art includes the following patents:

| Patent/Publication No. | | |
|---|---|---|
| (U.S. unless | | |
| stated otherwise) | Inventor | Issue/Publication Date |
| 2001/0038861 | Hsu et al. | 11-08-2001 |
| 2002/0051797 | Jezior | 05-02-2002 |
| 2002/0086039 | Lee et al. | 07-04-2002 |
| 2002/0192304 | Kennedy | 12-19-2002 |
| 2003/0092754 | Nishimuta et al. | 05-15-2003 |
| 4,005,191 | Clark | 01-25-1977 |
| 4,105,782 | Yu et al. | 08-08-1978 |
| 4,105,783 | Yu et al. | 08-08-1978 |
| 4,197,316 | Yu et al. | 08-08-1980 |
| 4,234,599 | Van Scott et al. | 11-18-1980 |
| 4,363,815 | Yu et al. | 12-14-1982 |
| 4,678,668 | Darras | 07-07-1987 |
| 5,091,171 | Yu et al. | 02-25-1992 |
| 5,543,149 | Rubin | 08-06-1996 |
| 5,554,654 | Yu et al | 09-10-1996 |
| 5,854,291 | Laughlin et al. | 12-29-1998 |
| 5,879,688 | Coury et al. | 03-09-1999 |
| 6,139,850 | Hahn et al. | 10-31-2000 |
| 6,217,885 | Roder et al. | 04-17-2001 |
| 6,368,579 | Barr | 04-09-2002 |
| 6,379,713 | Reiss | 04-30-2002 |
| 6,416,788 | Barr | 07-09-2002 |
| 6,506,375 | Barr | 01-14-2003 |
| 6,514,487 | Barr | 02-04-2003 |
| WO 96/29983 | Ramachandran et al. | 10-03-1996 |

Although the above cited art teaches anti-itch compositions containing various ingredients, none teach an anti-itch composition comprising an ammonium solution wherein water is used as a solvent and further comprising gelatin, a surfactant and menthol essential oils.

### SUMMARY OF THE INVENTION

The primary object of the present invention is to provide an anti-itch composition that neutralizes allergic reactions caused by insect bites.

A further object of the present invention is to provide an anti-itch composition that relieves the itching, swelling, pain and inflammation caused by insect bites.

An even further object of the present invention is to provide an anti-itch composition that may reduce anaphylaxis in some individuals.

Another object of the present invention is to provide an anti-itch composition that diminishes the severity of an allergic reaction.

Another object of the present invention is to provide an anti-itch composition that promotes tissue healing.

Another object of the present invention is to provide an anti-itch composition that has antiseptic properties.

A further object of the present invention is to provide an anti-itch composition that has bug repelling properties.

An even further object of the present invention is to provide an anti-itch composition that prevents secondary infections caused by insect bites.

An even further object of the present invention is to provide an anti-itch composition that has a pleasing fragrance.

The present invention fulfills the above and other objects by providing an anti-itch composition comprising an ammonium hydroxide solution, water, gelatin, a surfactant and menthol essential oils, wherein the ammonium hydroxide solution and water are the primary active ingredients. The first primary active ingredient, ammonium hydroxide, is a base which neutralizes the acidic substances found in insect bites, including formic acid which is found in red ant bites and bee stings. Because of the acid-base reaction, natural molecules of salt and water are formed, thereby neutralizing the allergic reaction and in fact reversing the reaction. The second primary active ingredient, water, is used to further neutralize and enhance the acid-base reaction. The secondary ingredients, gelatin, a surfactant and menthol essential oils, promote tissue healing and have antiseptic properties. In addition, the essential oils, which may be spearmint, peppermint, pennyroyal or eucalyptus, act as cooling agents and provide an aromatic fragrance. Citronella, cedarwood and pine oils may also be substituted for product variations, alone or in combination, for their bug repelling properties and aromatic fragrance.

The above and other objects, features and advantages of the present invention should become even more readily apparent to those skilled in the art upon a reading of the following detailed description.

### DESCRIPTION OF THE PREFERRED EMBODIMENTS

The anti-itch composition comprises the primary active ingredients ammonium hydroxide and water, as well as the secondary active ingredients gelatin, surfactant and menthol essential oils wherein the surfactant is preferably soap and the menthol essential oils may include, but is not limited to, spearmint, peppermint, pennyroyal or eucalyptus. The essential oils are utilized as an antiseptic cooling agent and for aromatic fragrance. In addition, citronella, cedarwood and pine oils, having bug repelling properties and aromatic fragrance, may be substituted to provide for product variations. The oils may be used individually or in combination to provide for various scents and insect repellant properties.

When a human or animal is bit by ants, bees or other insects, common reactions such as itching, swelling, redness, pain, inflammation and, in severe cases, anaphylaxis may occur. This is due to the insects injecting toxins into the human or animal. Such toxins are typically acids.

An acid is defined as “any of a class of chemical compounds whose aqueous solutions turn blue litmus paper red, react with and dissolve certain metals to form salts, and react with bases to form salts.” McGraw-Hill Dictionary of Scientific and Technical Terms, Sixth Edition, ©2003 The McGraw-Hill Companies, Inc.

Formic acid, also known as methanoic acid HCOOH, is a colorless, toxic, corrosive liquid with a sharp odor. Formic acid boils at 100.7 degrees Celsius and solidifies at 8.4 degrees Celsius. Formic acid has the lowest molecular weight and is the simplest of the carboxylic acids. Functionally, it is both an acid and an aldehyde. It reacts with most alcohols to form esters and decomposes when heated.

Ants and bees belonging to the order Hymenoptera inject formic acid antigens, which also stimulate the allergic response in humans and animals. Formic acid occurs in the bodies of red ants and in the stingers of bees and can be made by the oxidation of formaldehyde. This acidic antigen triggers the production of histamine and the allergic reactions that result therewith, including itching, redness, swelling, pain and anaphylaxis in hypersensitive individuals.

However, these allergic responses to acids from insects can be combated simply by utilizing a basic solution to ward off the acidic solutions. A base is defined as “any chemical species, ionic or molecular, capable of accepting or receiving a proton (hydrogen ion) from another substance; the other substance acts as an acid in giving of the proton.” Id.

When an acidic solution is combined with a basic solution, a neutralization reaction occurs wherein the acidic and basic ions in the solutions attempt to equalize each other. An example of an acid-base reaction is as follows:
1. When acid is a proton donor (Hcl)
   Hcl (aq)--->H⁺(aq)+Cl⁻
2. When base is OH donor (NaOH)
   NaOH (aq)--->Na⁺+OH⁻
3. Total Equation for Dissociation
   HCl (g)--->H⁺(aq)+Cl⁻(aq)
   NaOH (aq)--->Na⁺(aq)+OH⁻(aq)

An example of an acid-base reaction wherein water and salt molecules are yielded is as follows:
HCl (aq)+NaOH (aq)--->H₂O (l)+NaCl (aq)

Neutralization is as follows:
H₃O++Cl⁻+Na⁺ OH⁻--->2H₂O+Na⁺+Cl⁻

The H⁺ cation of the acid combines with the OH⁻ anion of the base to form water (H₂O) and the compound formed by the cation of the base and the anion of the acid is called a salt (NaCl). Salt is “the reaction product when a metal displaces the hydrogen of an acid” and applies to the products of all such acid-base reactions. Id.

Ammonia is a chemical compound whose molecule consists of one atom of nitrogen (N) and three atoms of hydrogen (H) with the chemical formula NH₃ and a structural formula of a tetrahedron with one empty corner. “In solution, [ammonia] forms the positively charged ammonium ion NH4⁺with a hydrogen atom on all four corners of the tetrahedron. At standard temperature and pressure, ammonia is a gas with a characteristic pungent small; its main uses are in the production of fertilizers, explosives and polymers.” The Columbia Encyclopedia, Sixth Edition, © 2003 Columbia University Press. An example of a chemical reaction between ammonia and water yielding ammonia and hydroxide is as follows:
NH₃+H₂O=NH₄+OH

Thus, the present invention utilizes chemical acid-base reactions to neutralize the allergic reactions caused by the acids from insects, fleas and the like. Each ingredient comprising the composition of the present invention is important as each performs a specific duty in ceasing the allergic reaction.

When applied to the site of the insect bite, the ammonium hydroxide acts as an antiseptic to cleanse the bite. The addition of the water increases the basal strength of the ammonium hydroxide. The gelatin and the surfactant binds the composition together and makes it thick and sticky so it will stay on a person's skin. In addition, the surfactant allows for ease of application to the skin as it smooths the texture of the composition. Finally, the essential oils not onlyprovide antiseptic, aromatherapic and bug repellent properties, but they also act as an extracting agent to pull the toxins out of a person.

In clinical trials, the composition of the present invention prevented an anaphylactic reaction on a hypersensitive individual who received single, double and triple simultaneous ant bites. Said individual previously experienced a severe anayphylaxis reaction after receiving multiple red ant bites on one occasion and experienced a severe outbreak of hives on another occasion after receiving a single bite from a carpenter red ant. Before the present invention, the allergic reactions caused from these types of bites on said individual have resulted in said individual seeking medical treatment, which has consisted of antihistamines, cortisone and epinephrine injections.

The composition of the present invention gave immediate relief from itching within 1-5 minutes and from redness and swelling within 2-10 minutes. In addition, the composition produced complete dissolution of the bite mark within 10-20 minutes and prevented secondary infection.

The preferred embodiment of the composition, measured by liquid volume, is as follows: ammonium hydroxide 30%; water 68%; gelatin 01%; menthol essential oil 0.05% and surfactant (soap) 0.05%.

Although the composition prevented an anaphylactic reaction for said individual, the composition is not a treatment for anaphylaxis. Thus, the composition is not a cure nor treatment for anaphylaxis.

Although only a few embodiments of the present invention have been described in detail hereinabove, all improvements and modifications to this invention within the scope or equivalents of the claims are included as part of this invention.

1. A liquid anti-itch composition comprising:
ammonium hydroxide;
water;
gelatin;
menthol essential oil; and
surfactant,
wherein an acid-base chemical reaction occurs to neutralize itchiness caused by insect, flea and bug bites when the composition is applied to a person's skin.

2. The anti-itch composition of claim 1 wherein:
said menthol essential oil is oil of spearmint.

3. The anti-itch composition of claim 1 wherein:
said menthol essential oil is oil of peppermint.

4. The anti-itch composition of claim 1 wherein:
said menthol essential oil is oil of pennyroyal.

5. The anti-itch composition of claim 1 wherein:
said menthol essential oil is citronella.

6. The anti-itch composition of claim 1 wherein:
said menthol essential oil is cedarwood.

7. The anti-itch composition of claim 1 wherein:
said menthol essential oil is eucalyptus.

8. The anti-itch composition of claim 1 wherein:
said menthol essential oil is pine oil.

9. The anti-itch composition of claim 1 wherein:
said surfactant is soap.

10. The anti-itch composition of claim 1 wherein:
said ammonium hydroxide is 30% by volume;
said water is 68% by volume;
said gelatin is 01% by volume;
said menthol essential oil is 0.05% by volume; and
said surfactant is 0.05% by volume.

11. The anti-itch composition of claim 1 wherein:
said acid-base chemical reaction prevents an anaphylactic reaction.

12. The anti-itch composition of claim 10 wherein:
said acid-base chemical reaction prevents an anaphylactic reaction.
